(19) United States
(12) Patent Application Publication
(10) Pub. No.: US 2026/0151728 A1
(43) Pub. Date: Jun. 4, 2026

(54) CONTAMINANT REMOVAL SYSTEMS

(71) Applicant: Honeywell International Inc., Charlotte, NC (US)

(72) Inventors: Phoebe Henson, Long Beach, CA (US); David Loeffelholz, Long Beach, CA (US); Rebecca Kamire, Buffalo Grove, IL (US); Zachery Clark, Sun City, AZ (US); Sam Butterwick, Chandler, AZ (US); Timothy Barber, Litchfield Park, AZ (US)

(21) Appl. No.: 19/094,674

(22) Filed: Mar. 28, 2025

Related U.S. Application Data
(60) Provisional application No. 63/726,398, filed on Nov. 29, 2024.

(51) Int. Cl. B01D 53/18; B01D 53/14; B01D 53/62; B01D 53/78; B01D 53/96; C07C 7/12

(52) U.S. Cl. CPC B01D 53/185; B01D 53/1425; B01D 53/1475; B01D 53/1493; B01D 53/62; B01D 53/78; B01D 53/96; C07C 7/12; B01D 2252/30; B01D 2257/504; B01D 2257/80; B01D 2258/06; B01D 2259/4575

(57) ABSTRACT

A contaminant removal device may be configured to transport a contaminant to an ionic liquid sorbent or from the ionic liquid sorbent. The contaminant removal device may include a housing include a liquid inlet configured to introduce the ionic liquid sorbent into the housing. The contaminant removal device may further include a packed bed configured to receive the ionic liquid sorbent and rotate about a device axis to centrifugally force the ionic liquid sorbent through the packed bed.

### CROSS-REFERENCE TO RELATED APPLICATIONS

The present application claims the benefit of U.S. Provisional Application Ser. No. 63/726,398, filed Nov. 29, 2024, which is incorporated herein by reference in entirety.

### TECHNICAL FIELD

The present disclosure relates to systems and techniques for removing contaminants from an environment using contaminant removal systems.

### BACKGROUND

An environmental control system (ECS) may provide conditioned air to a passenger cabin (e.g., of a spacecraft) or other environment. Some of this conditioned air may be treated to remove contaminants. ECS components that are used to remove contaminants may be large and heavy, increasing an overall weight of the ECS, and may consume large amounts of power heating, cooling, and pressurizing various fluid streams.

### SUMMARY

The disclosure describes systems and techniques for removing contaminants using an ionic liquid sorbent of an ionic liquid mixture using a rotating bed.

In some examples, the disclosure describes a contaminant removal device configured to transport a contaminant to an ionic liquid sorbent or from the ionic liquid sorbent. The contaminant removal device may include a housing including a liquid inlet configured to introduce the ionic liquid sorbent into the housing. The contaminant removal device may further include a packed bed configured to receive the ionic liquid sorbent and rotate about a device axis to centrifugally force the ionic liquid sorbent through the packed bed.

In some examples, the disclosure describes a contaminant removal system including a scrubber configured to absorb a contaminant from an air stream into an ionic liquid sorbent. The scrubber may include a scrubber housing, a gas inlet, a liquid inlet, and a scrubber packed bed. The gas inlet may be configured to introduce the air stream into the scrubber housing. The liquid inlet may be configured to introduce a liquid absorbent into the scrubber housing. The scrubber packed bed configured to receive the liquid absorbent from the liquid inlet and rotate about a scrubber axis to centrifugally force the liquid absorbent through the scrubber packed bed.

In some examples, the disclosure describes a method for removing a contaminant from an environment. The method may include absorbing, by a scrubber, a contaminant from an air stream into an ionic liquid sorbent centrifugally forced through a rotating scrubber packed bed in the scrubber. The method may further include transporting the ionic liquid sorbent comprising the contaminant to a stripper. The method may further include desorbing, by the stripper, the contaminant from the ionic liquid sorbent.

### BRIEF DESCRIPTION OF THE FIGURES

The details of one or more examples are set forth in the accompanying drawings and the description below. Other features, objects, and advantages will be apparent from the description and drawings, and from the claims.

FIG. 1 is a schematic diagram illustrating an example contaminant removal device configured to remove a contaminant.

FIG. 2 is a schematic diagram illustrating an example scrubber including a rotating bed and configured to remove a contaminant from an environment.

FIG. 3 is a schematic diagram illustrating an example stripper including a rotating bed and configured to remove a contaminant.

FIG. 4 is a block diagram illustrating an example contaminant removal system including a scrubber and a stripper for removing contaminants using an ionic liquid mixture.

FIG. 5 is a flow diagram illustrating an example method for removing a contaminant from an environment.

FIG. 6 is a schematic diagram illustrating an example contaminant removal system including a scrubber and a stripper.

### DETAILED DESCRIPTION

A contaminant removal system includes a scrubber that removes contaminants from an air stream, such as a cabin air of a spacecraft, through absorption using an ionic liquid sorbent in an ionic liquid mixture and an ionic liquid regeneration assembly (e.g., including a stripper) that desorbs the contaminants from the ionic liquid sorbent.

Contaminant removal systems described herein may be utilized as part of an environmental control system (ECS), such as in spacecraft, aircraft, watercraft, and the like. In some examples, contaminant removal systems may be used in an ECS of a resource-limited environment, such as a passenger cabin of a spacecraft, in which carbon dioxide and water may be recycled to produce oxygen gas, water, methane, and a variety of other compounds used in life support systems. Such resource-limited environments may be particularly suited for a contaminant removal system that includes components that use low amounts of power and have extended service lives to reduce overall weight, power consumption, and maintenance load.

Such contaminant removal systems may remove one or more of CO₂, trace contaminants, or humidity from air (e.g., from an environment) into an ionic liquid (e.g., in a scrubber), and then strip the CO₂, trace contaminants, or humidity from the ionic liquid (e.g., in a stripper). Certain contaminant removal systems use membranes in a scrubber and/or a stripper, to facilitate removal or extraction of one or more contaminants from a stream (e.g., from a gas stream such as cabin air or from a liquid stream such as that generated by a scrubber). However, membranes may experience fouling or wetting, especially when exposed to heat and high transmembrane pressure drop (e.g., in a stripper).

Contaminant removal systems, devices, and techniques according to the present disclosure use a rotating packed bed to facilitate contaminant removal. For example, a packed bed may be rotated within a reactor, and a stream may be forced centrifugally through the packed bed caused by rotation of the packed bed. Thus, a rotary or centrifugal packed bed may be used instead of, or in addition to, a membrane. A centrifugal packed bed may also promote performance since similar surface area/volume ratios can be achieved as a membrane while reducing or eliminating the membrane mass transfer coefficient.

In some examples, a contaminant removal system includes a scrubber and a stripper. One or both of the scrubber or the stripper may include a rotating or centrifugal packed bed reactor. The reactor may include an inner drum holding the packed bed and configured to rotate. The scrubber may include a gas inlet, a gas outlet, a liquid inlet and a liquid outlet. The gas inlet introduces cabin air in the scrubber. CO₂ in the cabin air is captured by a liquid absorbent (e.g., ionic liquid) within a packed bed (e.g., having a high surface area). The gas leaves with substantial reduction or elimination of CO₂. The liquid absorbent enters the scrubber through an inlet (e.g., a nozzle or a tube) and is picked up in the rotating packed bed reactor where CO₂ is absorbed. The centrifugal force of the rotating packed bed forces the liquid radially outward through the packed bed and to the outer edges of the inner drum. Further, the centrifugal force may pump the liquid from the outer edges through a pitot tube which is coupled to a liquid outlet of the reactor. Thus, CO₂ is removed from the cabin air to the liquid (e.g., ionic absorbent liquid) in the scrubber, and the freshened air may be recirculated to the cabin. Now, the liquid loaded with CO₂ is sent to a stripper to extract the CO₂ from the liquid.

The liquid flows through a heat exchanger and a heater and into the stripper. The stripper may also include a rotary packed bed reactor. Liquid follows a similar path through the stripper as in the scrubber. For example, the stripper may include a liquid inlet to introduce heated ionic liquid received from the scrubber, and the heated ionic liquid may be injected into a center of a rotating packed bed. The rotating packed bed picks up the injected liquid and spreads it across high surface area packing material as it spins. However, in the stripper, a gas inlet is not necessary, and only a gas outlet may be present (e.g., because gas is extracted from the CO₂-loaded liquid received from the scrubber).

Vacuum may be pulled at the gas outlet of the stripper to cause the CO₂ to be pumped out of the rotating packed bed (and ultimately, extracted from the CO₂-loaded liquid). For example, the vacuum encourages the release of CO2, water vapor and contaminants out of the ionic liquid. The spinning encourages the movement of the denser liquid to outer edges of the rotating packed bed. The rotating packed bed and inner drum forces the liquid to the outer edges of the drum where the high pressure caused by the spinning forces the liquid into a pitot tube. The pitot tube is coupled to a liquid outlet of the stripper. The freshened liquid exits the rotary stripper and is returned back to the scrubber, ready to extract CO₂ from cabin air in the scrubber. In some examples, both the scrubber and the stripper respectively include a rotary packed bed. In other examples, one of the scrubber and the stripper include a rotary packed bed, and the other of the scrubber and the stripper use membrane-based techniques to remove or extract CO₂.

Using a rotary bed may enable higher temperatures and/or pressures to be used in a stripper (e.g., because a membrane is no longer present, and typical membrane-limited conditions may be exceeded). Such higher temperatures and/or pressures may enable a relatively higher performance, e.g., compared to a membrane-based stripper. Further, eliminating the membrane may promote kinetics in both scrubbers and strippers. Further, a similar surface area for contacting liquid and/or gas with packed bed with can be achieved within a rotary bed.

The packed bed may include a substrate that is relatively easier to replace, maintain or clean compared to a membrane, and thus, rotary packed beds may extend a working life (e.g., between maintenance shut-downs, or an overall working life before disposal) of a scrubber or a stripper, compared to membrane-based systems.

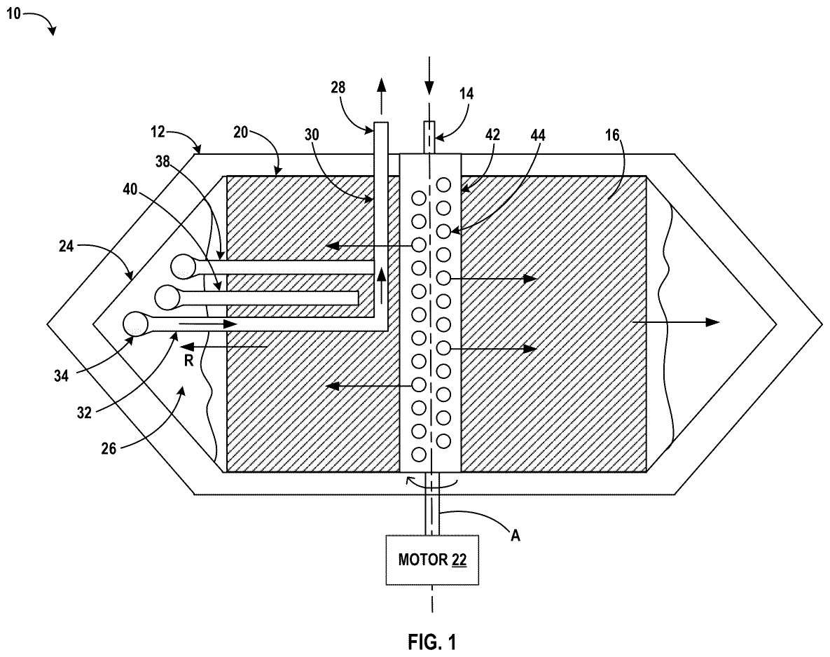

FIG. 1 is a schematic diagram illustrating an example contaminant removal device 10 configured to remove a contaminant. For example, contaminant removal device 10 may be configured to remove the contaminant from an environment, a chamber, a container, a vessel, a gas, or a liquid stream. In some examples, contaminant removal device 10 is configured to transport a contaminant to an ionic liquid sorbent or from the ionic liquid sorbent (for example, one of transport the contaminant to the ionic liquid sorbent or transport the contaminant from the ionic liquid sorbent). An ionic liquid mixture may include the ionic liquid sorbent, and the contaminant may thus be ultimately transported to or from the ionic liquid mixture. In some examples, the contaminant includes carbon dioxide.

Contaminant removal device 10 includes a housing 12. Housing 12 may have any suitable geometric shape. Housing 12 may include any suitable material, for example, a metal or an alloy. Housing 12 may include a liquid inlet 14 configured to introduce the ionic liquid sorbent (e.g., the ionic liquid mixture including the ionic liquid sorbent) into housing 12.

The contaminant removal device may further include a packed bed 16 configured to receive the ionic liquid sorbent (e.g., from liquid inlet 14) and rotate about a device axis A to centrifugally force the ionic liquid sorbent through packed bed 16 (e.g., in a radial direction R shown in FIG. 1). Packed bed 16 may include any suitable packing material configured to promote contact between a gas stream and a liquid stream (e.g., between air and ionic liquid sorbent in a scrubber) or to promote release of contaminant from a liquid stream (e.g., release of gaseous contaminant from contaminant-loaded ionic liquid sorbent in a stripper). The packing material may include one or more of a foam, beads, a gel, a woven material, a non-woven material, or any combination thereof. The packing material may include one or more of an organic material, an inorganic material, a metal, an alloy, or a ceramic. In some examples, the packing material includes an additively manufactured cellular structure. The packing material may be random packed, or have a predetermined structure.

In some examples, housing 12 is a first housing, and contaminant removal device 10 further includes a second housing 20 within an interior of first housing 12. Second housing 20 may be configured to retain packed bed 16. Second housing 20 may include any suitable material, for example, same or different from that of first housing 20. Second housing 20 may be fluid tight to retain liquid introduced via liquid inlet 14 within second housing 20. Second housing 12 may have any suitable geometric configuration, for example, to receive and hold a volume of liquid and packed bed 16. In some examples, second housing 12 has a generally cylindrical configuration. In some examples, the cylindrical configuration may exhibit a variation in radius. For example, the outermost radius of housing 20 may increase in a direction along device axis A from opposing ends of housing 20 to a center of housing 20. In some examples, at least a portion of housing 20 defines a truncated cone. In some examples, housing 20 defines two truncated cones.

First housing 12 may be configured to maintain a fixed position, and second housing 20 may be configured to rotate relative to first housing 12 and about device axis A to cause packed bed 16 to rotate about device axis A. For example, an axle may extend along contaminant removal device 10, and second housing 20 may be mechanically coupled to the axle and configured to rotate with the axis. In some examples, contaminant removal device 10 further includes a motor 22 mechanically coupled to second housing 20 and configured to rotate second housing 20. For example, motor 22 may be coupled to second housing 20 by a shaft (e.g., of the axle), and cause second housing 20 to rotate at any suitable predetermined speed. For example, the speed may be configured to be sufficient to result in a centrifugal transport of liquid introduced into second housing 20 via liquid inlet 14 through packed bed 16.

In some examples, second housing 20 defines a circumferential bulge 24 radially outward of packed bed 16 relative to device axis A. The circumferential bulge 24 defines a collection region 26 configured to receive the ionic liquid sorbent centrifugally forced through packed bed 16. Thus, the ionic liquid sorbent may be received within second housing 20 from inlet 14 and centrifugally forced (by rotation of second housing 20) through packed bed 16 to collection region 26.

Housing 12 may further include a liquid outlet 28. For example, ionic liquid sorbent (e.g., the ionic liquid mixture including the ionic liquid sorbent) may be centrifugally forced through packed bed 16 and transported to liquid outlet 28. In some examples, liquid outlet 28 is fluidically coupled to collection region 26. For example, an outlet conduit 30 may fluidically couple collection region 26 and liquid outlet 28. For example, outlet conduit 30 may including one or more sections of piping. The sections of piping be positioned along any suitable path.

In some examples, contaminant removal device 10 further includes a pitot tube 32 configured to transport ionic liquid sorbent centrifugally forced through the packed bed 16 to liquid outlet 28. For example, pitot tube 32 may be coupled to or form a first end of outlet conduit 30, and liquid outlet 28 may be coupled to or form a second end of outlet conduit 30.

In some examples, pitot tube 32 defines a pitot inlet 34 positioned in collection region 26 radially outward of packed bed relative to device axis A. Pitot inlet 34 is configured to introduce at least a portion of the ionic liquid sorbent from collection region 26 into pitot tube 32. Pitot inlet 34 (or another pitot inlet of another pitot tube) may include an inlet funnel, or a scoop.

In some examples, pitot tube 34 is a first pitot tube, and contaminant removal device 10 further includes a second pitot tube 38 radially inward relative to first pitot tube 36. Second pitot tube 38 may be configured to introduce at least a portion of the ionic liquid sorbent from an overflow region (e.g., within or adjacent collection region 26) into second pitot tube 38.

In some examples, contaminant removal device 10 further includes a third pitot tube 40 radially between first pitot tube 34 and second pitot tube 38, and configured to recirculate the ionic liquid sorbent. For example, third pitot tube 40 may be configured to recirculate the ionic liquid sorbent from collection region 26 to another region in second housing 20 (e.g., radially inward to packing bed 16 or any other region). The recirculation may cause the ionic liquid sorbent to flow through packing bed 16 multiple times, which might promote or facilitate progressive removal of residual contaminant from the ionic liquid sorbent.

Contaminant removal device 10 may further include a liquid distribution conduit 42 coupled to liquid inlet 14 and extending along device axis A. Liquid distribution conduit 42 may have any suitable shape or geometry, for example, an elongated or cylindrical geometry. Liquid distribution conduit 42 may include any suitable material, for example, the same as or different from those of first housing 12 or second housing 20. Liquid distribution conduit 42 may define a plurality of liquid distribution openings 44 configured to distribute the ionic liquid sorbent received from liquid inlet 14 to packed bed 16. In some examples, plurality of liquid distribution openings 44 includes at least one opening defined by a body of liquid distribution conduit 42 (e.g., a perforation). In some examples, plurality of liquid distribution openings 44 includes at least one nozzle fluidically coupled to liquid distribution conduit 42. Liquid distribution openings 44 may have any suitable peripheral shape, including circular, elliptical, polygonal, curved, or combinations thereof. In some examples, liquid distribution openings 44 are circular. Each of liquid distribution openings 44 may be identical to, or differ from, other liquid distribution openings, in one or both of size or shape.

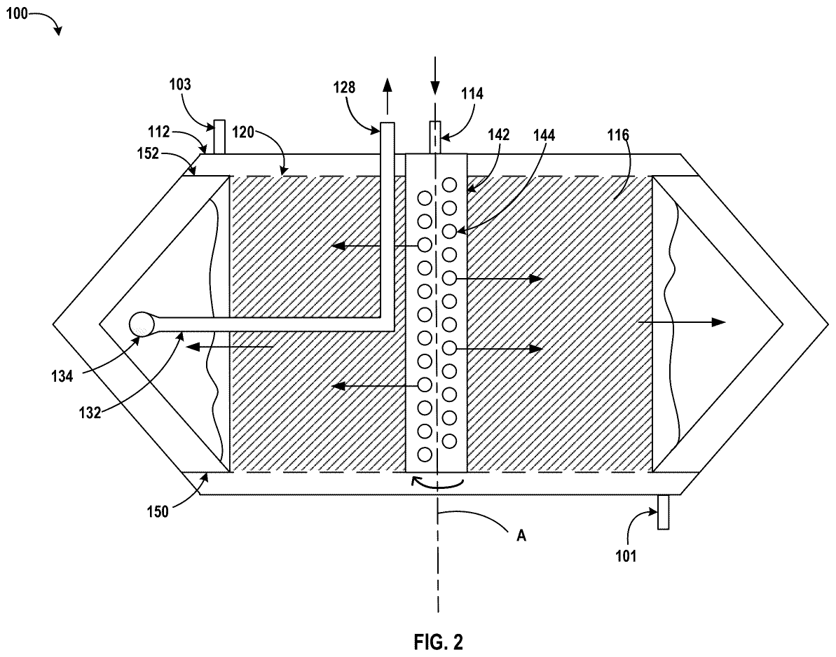

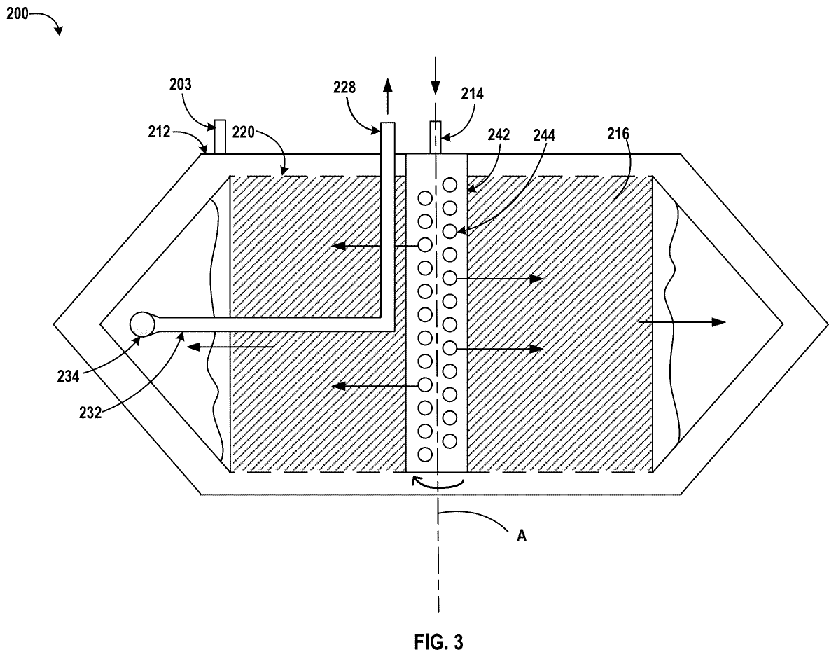

In some examples, contaminant removal device 10 (or a similar device) may be used as a scrubber (as described with reference to FIG. 2), or as a stripper (as described with reference to FIG. 3), in a contaminant removal system.

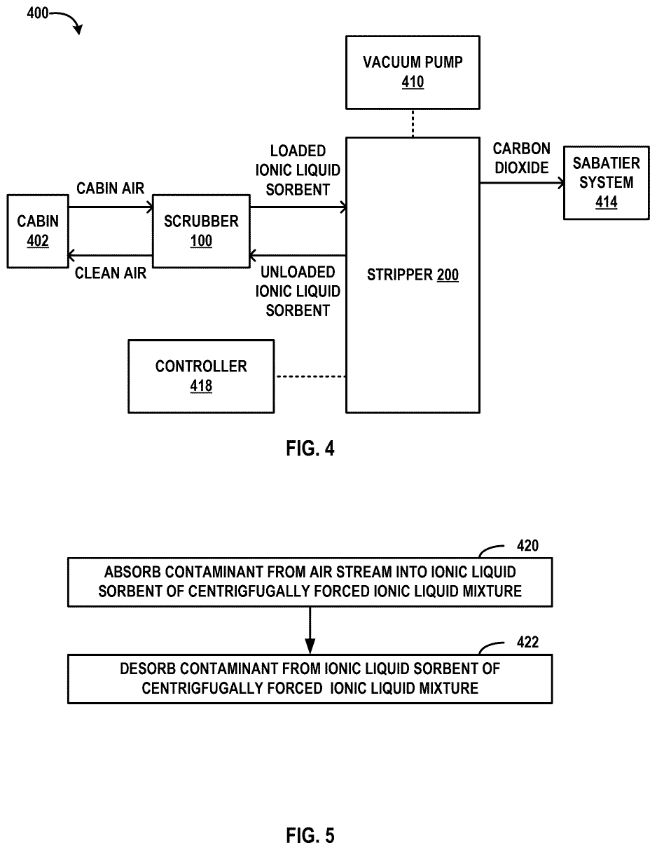

FIG. 2 is a schematic diagram illustrating a scrubber 100 configured to remove a contaminant. Scrubber 100 may be used in a contaminant removal system (e.g., as described with reference to FIGS. 4 to 6). Scrubber 100 may be similar to contaminant removal device 10 (except for the differences described with reference to FIG. 2), and respective component of scrubber 100 are numbered similar to like components of contaminant removal device 10.

Scrubber 100 may be configured to absorb a contaminant from an air stream into an ionic liquid sorbent. Scrubber 100 further comprising a gas inlet 101 configured to introduce the air stream into scrubber 100. Scrubber 100 includes a first scrubber housing 112. Scrubber 100 further includes a gas inlet 101 configured to introduce the air stream into first scrubber housing 112. For example, gas inlet 101 may be configured to introduce the air stream from a cabin into scrubber 100. Scrubber 100 further includes a liquid inlet 114 configured to introduce the ionic liquid sorbent into scrubber 100 (e.g., into first scrubber housing 112).

Scrubber 100 further includes a scrubber packed bed 116 configured to receive the ionic liquid sorbent from liquid inlet 114 and rotate about a scrubber axis A to centrifugally force the ionic liquid sorbent through scrubber packed bed 116. For example, scrubber 100 may include a second scrubber housing 120 configured to hold scrubber packed bed 116 and rotate scrubber packed bed 116 (e.g., about scrubber axis A and relative to first scrubber housing 116). Scrubber 100 may further include a gas outlet 103 configured to discharge a processed air stream from which the contaminant is at least partially scrubbed by scrubber 100. For example, gas outlet 103 may discharge decontaminated cabin air, for transport back to a cabin.

Scrubber 100 may include one or more pitot tubes. For example, scrubber 100 may include a pitot tube 132 having a pitot inlet 134. Scrubber 100 may include additional pitot tubes (e.g., similar to pitot tubes 38, and/or 40 described with reference to FIG. 1). Scrubber 100 further includes liquid outlet 128 (fluidically coupled to pitot inlet 134) configured to discharge ionic liquid (e.g., contaminant-loaded ionic liquid) after it is circulated or recirculated through scrubber packed bed 116. Thus, scrubber 100 may be configured to decontaminate an air stream (e.g., from a cabin) introduced into gas inlet 101, by contacting the air stream with the ionic liquid sorbent in scrubber packed bed 116, and discharging the decontaminated air stream via gas outlet 103. Scrubber 100 may further include a liquid distribution conduit 142 and a plurality of liquid distribution outlets 144 configured to distribute the ionic liquid sorbent through scrubber packed bed 116. Likewise, fresh ionic liquid sorbent (e.g., having at least some capacity to absorb a contaminant) may be introduced into scrubber 100 through liquid inlet 114, and contaminant-loaded ionic liquid sorbent may be discharged through liquid outlet 128.

Scrubber 100 may further include at least one seal. For example, the at least one seal may include a first seal 150 adjacent gas inlet 101 and a second seal 152 adjacent gas outlet 103. First seal 150 and second seal 152 may prevent migration or transport of gas directly from gas inlet 101 to gas outlet 103 without being treated by ionic liquid sorbent in scrubber packed bed 116.

FIG. 3 is a schematic diagram illustrating a stripper 200 configured to remove a contaminant. Stripper 200 may be used in a contaminant removal system (e.g., as described with reference to FIGS. 4 to 6). Stripper 200 may be similar to contaminant removal device 10 or scrubber 100 (except for the differences described with reference to FIG. 3), and respective components of stripper 200 are numbered similar to like components of contaminant removal device 10 or scrubber 100.

For example, stripper 200 may be configured to desorb the contaminant from the ionic liquid sorbent. Stripper 200 may include a first stripper housing 212 including a liquid inlet 214 configured to introduce the ionic liquid absorbent (e.g., contaminant-loaded ionic liquid sorbent) received from liquid outlet 103 of scrubber 100 into first stripper housing 212. Stripper 200 may further include a stripper packed bed 216 configured to receive the liquid absorbent from liquid inlet 214 and rotate about a stripper axis A to centrifugally force the liquid absorbent through stripper packed bed 216. For example, stripper 200 may include a second stripper housing 220 configured to hold stripper packed bed 216 and rotate stripper packed bed 216 (e.g., about stripper axis A and relative to first stripper housing 216). Stripper 200 may further include a gas outlet 203 configured to discharge a gas stream including the contaminant at least partially stripped by stripper 200 (e.g., from the ionic liquid sorbent introduced via liquid inlet 214). For example, gas outlet 203 may discharge the contaminant for further processing. In examples in which the contaminant includes CO₂, gas outlet 203 may be fluidically coupled to a Sabatier reactor.

Stripper 200 may include one or more pitot tubes. For example, stripper 200 may include a pitot tube 232 having a pitot inlet 234. Stripper 200 may include additional pitot tubes (e.g., similar to pitot tubes 38, and/or 40 described with reference to FIG. 1). Stripper 200 further includes liquid outlet 228 (fluidically coupled to pitot inlet 234) configured to discharge ionic liquid (e.g., contaminant-reduced ionic liquid) after it is circulated or recirculated through scrubber packed bed 216. Stripper 200 may further include a liquid distribution conduit 242 and a plurality of liquid distribution outlets 244 configured to distribute the ionic liquid sorbent through stripper packed bed 216. Stripper 200 may further include at least one seal. Thus, stripper 200 may be configured to unload contaminant from a contaminant-loaded ionic liquid (e.g., received from a scrubber) introduced into liquid inlet 214, by contacting the liquid sorbent with stripper packed bed 216, and discharging the unloaded ionic liquid via liquid outlet 228.

FIG. 4 is a block diagram illustrating an example contaminant removal system 400 including scrubber 100, stripper 200, and a vacuum pump 410 for removing contaminants from an environment. Contaminant removal system 400 is configured to remove contaminants from a cabin 402. Cabin 402 may be a controlled environment, such as an aircraft cabin, spacecraft cabin, watercraft cabin, or the like, and contaminants removed from cabin 402 may include, but are not limited to, carbon dioxide, water, hydrocarbons, permanent gases, or the like. In the example of FIG. 4, cabin 402 is a cabin of a closed-loop system, such as a spacecraft cabin or submarine cabin, in which components of a cabin air stream from cabin 402, such as carbon dioxide and water, may be removed within contaminant removal system 400, allowing a purified supply air stream to be generated and carbon dioxide and water to be recovered. However, in other examples, cabin 402 may be a cabin of an open-loop system, such as an aircraft cabin, in which components of a cabin air stream may be removed to generate a purified supply air stream with only partial or no subsequent recovery of the contaminants.

In examples in which the contaminant includes carbon dioxide, contaminant removal system 400 may include a Sabatier system 414 configured to convert the carbon dioxide to methane. Sabatier system 414 may be configured to react the contaminant with hydrogen to produce water and methane and either dry the methane for storage as fuel or send the methane to other reactors for further conversion. For example, while not shown in FIG. 4, system 400 may include a methane pyrolysis system configured to convert methane to carbon and hydrogen, and subsequently return at least a portion of the hydrogen to Sabatier system 414 to produce more methane and water. Sabatier system 414 may be configured to send the water to an electrolysis system to produce hydrogen and oxygen, contributing to a more closed loop O₂/CO₂ cycle. In other examples, Sabatier system 414 may further pressurize the methane to above ambient pressure to form methane for rocket fuel.

System 400 is configured to remove the contaminants using an ionic liquid sorbent. Ionic liquid sorbents may be salts that are generally comprised of an anion and an organic cation. These salts may be liquid at their temperature of use, have effectively zero vapor pressure, be generally nontoxic, and/or have sufficient stability to resist deterioration. In some examples, liquid sorbents may contain relatively large organic cations and any of a variety of anions, which may be tailored to obtain desired characteristics, such as characteristics that improve absorption of the contaminant under operating conditions of system 400 and/or exclusion of the ionic liquid sorbent by a filtration membrane. Ionic liquid sorbents may be water soluble, hygroscopic (i.e., capable of absorbing moisture from the air), and/or capable of releasing water by evaporation, such as by elevating the temperature or reducing the water partial pressure. In system 400, the ionic liquid sorbent is dissolved in water to form an ionic liquid mixture. A mass fraction (or concentration) of ionic liquid sorbent in the ionic liquid mixture may be sufficiently high to remove contaminants and sufficiently low that the ionic liquid sorbent remains in solution through operating ranges (e.g., temperature range, pH range) and/or maintains a low viscosity.

Contaminant removal system 400 includes scrubber 100. Scrubber 100 is configured to absorb one or more contaminants from a cabin air stream into the ionic liquid sorbent and discharge a clean air stream to cabin 102. Scrubber 100 is configured to receive unloaded ionic liquid sorbent having a relatively low concentration of contaminants, contact the unloaded ionic liquid sorbent with cabin air through one or more separation membranes to absorb the contaminants into the ionic liquid sorbent, and discharge loaded ionic liquid sorbent that includes a higher concentration of contaminants.

Contaminant removal system 400 includes a stripper 200 configured to desorb one or more contaminants from the loaded ionic liquid sorbent and discharge the contaminants from stripper 200, such as to a Sabatier system 414. Stripper 200 is configured to receive the loaded ionic liquid sorbent from scrubber 100, contact the loaded ionic liquid sorbent with a sweep gas stream or other low pressure volume through one or more separation membranes to desorb the contaminant from the ionic liquid sorbent, and discharge unloaded liquid sorbent that includes a lower concentration of the contaminant. To desorb the contaminant from the ionic liquid sorbent, the gas phase side of stripper 200 may be at a vacuum. Contaminant removal system 400 may further include a vacuum pump 410 configured to generate the vacuum to desorb the contaminant from the ionic liquid sorbent and pressurize the contaminant, such as for further storage or processing.

Contaminant removal system 400 may include a process control system that includes a controller 418 and one or more sensor sets (not shown). Controller 418 may be configured to receive measurements from the one or more sensor sets and/or components of contaminant removal system 400 and/or send control signals to components of contaminant removal system 104. Controller 418 may be communicatively coupled to and configured to receive measurement signals from the one or more sensor sets, and other process control components (not shown) of contaminant removal system 400, such as: control valves for various streams; pumps; heaters; heat exchangers; compressors; and the like. The sensor sets may include instrumentation configured to detect any of a pressure, temperature, flow rate, and/or contaminant concentration (e.g., carbon dioxide concentration or water concentration) of a liquid or gas stream of contaminant removal system 400. Controller 418 may be configured to use the detected conditions to control operation of contaminant removal system 400 to function as described in the application.

Controller 418 may include any of a wide range of devices, including control circuitry, processors (e.g., one or more microprocessors, one or more application specific integrated circuits (ASICs), one or more field programmable gate arrays (FPGAs), or the like), processing circuitry, one or more servers, one or more desktop computers, one or more notebook (i.e., laptop) computers, one or more cloud computing clusters, or the like.

In some examples, controller 418 is configured to control a contaminant concentration within the environment of cabin 402. For example, controller 418 may be configured to receive a contaminant concentration measurement for a contaminant, such as from cabin air sensor set or a contaminant concentration sensor in cabin 402. Controller 418 may be configured to determine whether the contaminant concentration measurement exceeds a contaminant concentration setpoint. For example, the contaminant concentration setpoint may be a target concentration of the supply air stream for maintaining cabin 402 below a threshold contaminant concentration. Controller 418 may be configured to send, in response to the contaminant concentration measurement exceeding the contaminant concentration setpoint, a control signal to decrease a concentration of the contaminant in the supply air stream. For example, controller 418 may send a control signal to control a flow rate of the ionic liquid mixture; a flow rate, humidity, and/or temperature of a sweep gas stream into stripper 200; a temperature of the ionic liquid mixture at scrubber 100 or stripper 200; a flow rate of the cabin air stream; or any other variable that may control a rate of removal of contaminants from the cabin air stream.

FIG. 5 is a flow diagram illustrating an example method for removing a contaminant from an environment, described with respect to system 400 of FIG. 4. However, the method of FIG. 5 may be performed using any suitable system.

To remove contaminants from cabin 402, the method includes absorbing, by scrubber 100, a contaminant from an air stream into an ionic liquid sorbent in an ionic liquid mixture (420). For example, scrubber 100 may centrifugally force a contaminant from an air stream into an ionic liquid sorbent through rotating scrubber packed bed 116 in scrubber 100. Controller 418 may detect that the cabin air stream has a concentration of one or more contaminants that is above a threshold and, in response, control components of a cabin air circuit to control a flow rate of the cabin air stream and components of an ionic liquid circuit to control a flow rate and temperature of the ionic liquid mixture. As a result, a clean air stream has a lower concentration of the contaminant than the cabin air stream. For example, the clean air stream may have a concentration of a contaminant that is about 25% to about 99% less than a concentration of the contaminant in the cabin air stream.

To recover the contaminant from the ionic liquid sorbent, the method includes desorbing, by stripper 200, the contaminant from the ionic liquid sorbent (422). For example, the ionic liquid sorbent including the contaminant may be transported to stripper 200. The method may further include desorbing, by stripper 200, the contaminant from the ionic liquid sorbent. For example, controller 418 may control components of an ionic liquid circuit to control a flow rate of ionic liquid sorbent between scrubber 100 and stripper 200 and control components for processing a contaminant stream, such as vacuum pump 410, to create a pressure differential across stripper 200 to cause stripper 200 to desorb the contaminant from the ionic liquid sorbent.

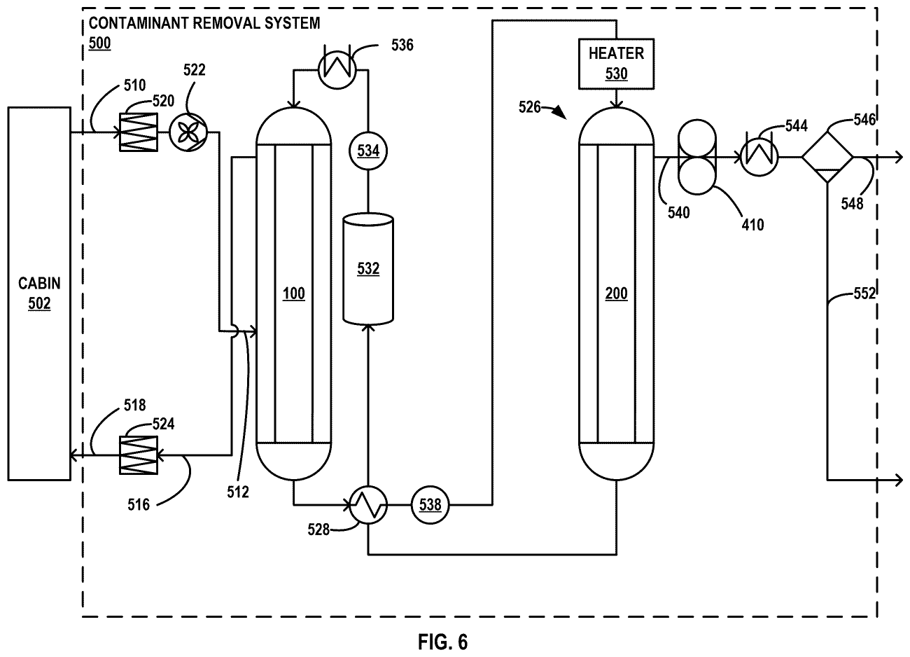

FIG. 6 is a schematic diagram illustrating an example contaminant removal system 500 that includes vacuum pump 410 between scrubber 100 and stripper 200. Contaminant removal system 200 includes a cabin air circuit configured to circulate cabin air between a cabin 502 and scrubber 100. In the example of FIG. 6, cabin air stream 410 includes a filter 520 configured to remove particulates from cabin air stream 510 prior to entry into scrubber 100 and a blower 522 configured to draw cabin air into scrubber 100, while a supply air stream 518 includes a filter 224 configured to remove any leaked ionic liquid sorbent and/or further filter clean air from supply air stream 518 prior to entry into cabin 102.

Contaminant removal system 500 includes an ionic liquid circuit 526 configured to circulate ionic liquid sorbent between scrubber 100 and stripper 200. For example, a pump 534 may pump unloaded ionic liquid sorbent from an ionic liquid storage 532 and/or stripper 200 into scrubber 100. Unloaded ionic liquid sorbent may include unused ionic liquid sorbent free of contaminants or regenerated ionic liquid sorbent having a lower concentration of contaminants than the loaded ionic liquid sorbent. In some examples, the unloaded ionic liquid sorbent may be cooled by a cooler 536 prior to entry into scrubber 100. In some examples, the loaded ionic liquid sorbent may be pumped by a pump 538 and/or preheated by a heat exchanger 528 and/or heater 530 prior to entry into stripper 200.

Scrubber 100 is configured to absorb the contaminant from cabin air stream 512 into an ionic liquid sorbent and discharge a clean air stream 516 to cabin 502. On a gas phase side, scrubber 100 is configured to receive cabin air from cabin air stream 512 that includes contaminant species from cabin 502, such as carbon dioxide, water, hydrocarbon volatiles, permanent gases, and other gaseous substances. Scrubber 100 is configured to absorb one or more contaminant species in the cabin air into an ionic liquid sorbent. Clean air from clean air stream 516 discharged from scrubber 100 may have a lower concentration of contaminants than cabin air from cabin air stream 512 received by scrubber 100. Scrubber 100 is configured to discharge a clean air stream 516 to cabin 502.

On a liquid phase side, scrubber 100 is configured to receive unloaded ionic liquid sorbent, such as from ionic liquid storage 532. The unloaded ionic liquid sorbent may flow through scrubber 100 and absorb contaminants from cabin air through the scrubber packed bed 116 of scrubber 100. As a result, the loaded ionic liquid sorbent discharged from scrubber 100 may have a higher concentration of contaminants than the unloaded ionic liquid sorbent received by scrubber 100. Scrubber 100 may discharge the loaded ionic liquid sorbent containing the contaminants to stripper 200.

Stripper 200 is downstream of scrubber 100. Stripper 200 is configured to desorb the contaminant from the ionic liquid sorbent into a contaminant stream 540. On a liquid phase side, stripper 200 is configured to receive loaded ionic liquid sorbent from scrubber 100 and desorb one or more contaminants from the loaded ionic liquid sorbent. Unloaded ionic liquid sorbent discharged from stripper 200 may have a lower concentration of contaminants than the loaded ionic liquid sorbent received by stripper 200. On a gas phase side, stripper 200 is configured to discharge the contaminant in a contaminant stream 540. Contaminant stream 540 may be continuously removed from stripper 200 to assist migration of the contaminants from the loaded ionic liquid sorbent into contaminant stream 540.

Scrubber 100 and/or stripper 200 may include a rotating packed bed (as described with reference to FIGS. 1 to 4), or one or more membrane separators configured to flow air on a first side and flow ionic liquid sorbent on a second, opposite side.

Vacuum pump 410 is configured to generate the vacuum in stripper 200 to drive or facilitate desorption of contaminant. Vacuum pump 410 is positioned downstream of scrubber 100 and upstream of stripper 200.

In the example of FIG. 6, contaminant removal system 500 may include one or more systems or components configured to further process contaminant stream 540. In some examples, contaminant removal system 500 includes a compressor (not shown), condenser 544, and water separator 546 configured to compress and/or condense contaminant stream 540 and remove water from the compressed or condensed contaminant stream 540. For example, for carbon dioxide removed from contaminant removal system 500 to be stored or recycled, the compressor, condenser 544, and water separator 546 may compress contaminant stream 540 to a high pressure and remove nearly all water from contaminant stream 540. In a life support application, a large amount of water may be present in cabin air stream 510. For example, the concentration of water in cabin air stream 510 may be much higher than that of carbon dioxide. Sabatier system 514 may require a water concentration of less than 2% to react hydrogen gas with carbon dioxide.

Contaminant stream 540 may be compressed by a compressor. A variety of compressors may be used including, but not limited to, centrifugal compressors, positive displacement compressors, and the like. Condenser 544 may be configured to cool contaminant stream 540 (e.g., after compression) and condense water from contaminant stream 540. For example, condenser 544 may be coupled to a refrigeration system or other cooling system that circulates a cooling medium to cool contaminant stream 540. A variety of condensers may be used for condenser 544 including, but not limited to, shell and tube heat exchangers, plate-fin, surface coolers, heat pipes, thermoelectric devices, cooling jackets, and the like. Water separator 546 may be configured to remove water from contaminant stream 540, discharge a dehumidified contaminant stream 548 to Sabatier system 514, and discharge contaminant water stream 552 to water storage 516. A variety of water separators may be used including, but not limited to, static phase separators, capillary phase separator, membrane phase separators, centrifugal/rotary separators, and the like.

Controller 418 may be communicatively coupled to and configured to receive measurement signals from the one or more sensor sets, and other process control components (not shown) of contaminant removal system 500, such as: vacuum pump 410; control valves for cabin air stream 510, clean air stream 516, supply air stream 518, contaminant stream 540, and inlets/outlets to heat exchanger 528, heater 530, ionic liquid storage 532, and cooler 536; pump 534; pump 538; blower 522; and the like.

The following enumerated clauses describe examples according to the present disclosure.

Clause 1: A contaminant removal device configured to remove a contaminant to an ionic liquid sorbent or from the ionic liquid sorbent, the contaminant removal device including: a housing including a liquid inlet configured to introduce the ionic liquid sorbent into the housing; and a packed bed configured to receive the ionic liquid sorbent and rotate about a device axis to centrifugally force the ionic liquid sorbent through the packed bed.

Clause 2: The contaminant removal device of clause 1, where the housing is a first housing, the device further including a second housing within an interior of the first housing, where the second housing is configured to retain the packed bed, where the first housing is configured to maintain a fixed position; and where the second housing is configured to rotate relative to the first housing and about the device axis to cause the packed bed to rotate about the device axis.

Clause 3: The contaminant removal device of clause 2, further including a motor mechanically coupled to the second housing and configured to rotate the second housing.

Clause 4: The contaminant removal device of clauses 2 or 3, where the second housing defines a circumferential bulge radially outward of the packed bed relative to the device axis, where the circumferential bulge defines a collection region configured to receive the ionic liquid sorbent centrifugally forced through the packed bed.

Clause 5: The contaminant removal device of any of clauses 1 to 4, where the housing further includes a liquid outlet, the device further including a pitot tube configured to transport centrifugally forced ionic liquid sorbent from the packed bed to the liquid outlet.

Clause 6: The contaminant removal device of clause 5, where the pitot tube defines a pitot inlet positioned in a collection region radially outward of the packed bed relative to the device axis, and where the pitot inlet is configured to introduce at least a portion of the ionic liquid sorbent from the collection region into the pitot tube.

Clause 7: The contaminant removal device of clause 6, where the pitot inlet includes an inlet funnel.

Clause 8: The contaminant removal device of any of clauses 5 to 7, where the pitot tube is a first pitot tube, and where the contaminant removal device further includes: a second pitot tube radially inward relative to the first pitot tube and configured to introduce at least a portion of the ionic liquid sorbent from an overflow region into the second pitot tube; and a third pitot tube radially between the first pitot tube and the second pitot tube and configured to recirculate the ionic liquid sorbent.

Clause 9: The contaminant removal device of any of clauses 1 to 8, further including a liquid distribution conduit coupled to the liquid inlet and extending along the device axis, where the liquid distribution conduit defines a plurality of liquid distribution openings configured to distribute the ionic liquid sorbent received from the liquid inlet to the packed bed.

Clause 10: The contaminant removal device of any of clauses 1 to 9, where the contaminant removal device is a scrubber configured to absorb the contaminant from an air stream into the ionic liquid sorbent, the scrubber further including a gas inlet configured to introduce the air stream into the scrubber.

Clause 11: The contaminant removal device of clause 10, further including a gas outlet configured to discharge a processed air stream from which the contaminant is at least partially scrubbed by the scrubber.

Clause 12: The contaminant removal device of any of clauses 1 to 9, where the contaminant removal device is a stripper configured to desorb the contaminant from the ionic liquid sorbent, the stripper further including a liquid outlet configured to discharge a processed liquid stream from which the contaminant is at least partially stripped by the stripper.

Clause 13: The contaminant removal device of clause 12, further including a gas outlet configured to discharge a gas stream including the contaminant at least partially stripped by the stripper.

Clause 14: A contaminant removal system, including: a scrubber configured to absorb a contaminant from an air stream into an ionic liquid sorbent, the scrubber including: a scrubber housing; a gas inlet configured to introduce the air stream into the scrubber housing; a liquid inlet being configured to introduce the ionic liquid sorbent into the scrubber housing; and a scrubber packed bed configured to receive the ionic liquid sorbent from the liquid inlet and rotate about a scrubber axis to centrifugally force the ionic liquid sorbent through the scrubber packed bed.

Clause 15: The contaminant removal system of clause 14, further including: a stripper configured to desorb the contaminant from the ionic liquid sorbent, the stripper including: a stripper housing including a liquid inlet configured to introduce the ionic liquid absorbent received from the liquid outlet of the scrubber into the stripper housing; and a stripper packed bed configured to receive the ionic liquid absorbent from the liquid inlet and rotate about a stripper axis to centrifugally force the ionic liquid absorbent through the stripper packed bed.

Clause 16: The contaminant removal system of clause 14, where the contaminant is carbon dioxide.

Clause 17: The contaminant removal system of clause 16, further including a Sabatier system coupled to the ionic liquid regeneration assembly and configured to generate hydrocarbons using the contaminant.

Clause 18: A method for removing a contaminant from an environment, the method including: absorbing, by a scrubber, a contaminant from an air stream into an ionic liquid sorbent centrifugally forced through a rotating scrubber packed bed in the scrubber; transporting the ionic liquid sorbent including the contaminant to a stripper; and desorbing, by the stripper, the contaminant from the ionic liquid sorbent.

Clause 19: The method of clause 18, where the desorbing further includes centrifugally forcing the ionic liquid sorbent through a rotating stripper packed bed in the stripper.

Clause 20: The method of clauses 18 or 19, where the contaminant is carbon dioxide, the method further comprising generating, by a Sabatier system, hydrocarbons using the desorbed contaminant.

The techniques described in this disclosure may be implemented, at least in part, in hardware, software, firmware, or any combination thereof. For example, various aspects of the described techniques, such as functionality attributed to controller 118 or the various sensors above, may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components, or integrated within common or separate hardware, firmware, or software components.

The techniques described in this disclosure may also be embodied or encoded in an article of manufacture including a computer-readable storage medium encoded with instructions. Instructions embedded or encoded in an article of manufacture including a computer-readable storage medium, may cause one or more programmable processors, or other processors, to implement one or more of the techniques described herein, such as when instructions included or encoded in the computer-readable storage medium are executed by the one or more processors. Computer readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

In some examples, a computer-readable storage medium may include a non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described. These and other examples are within the scope of the following claims.

What is claimed is:

1. A contaminant removal device configured to remove a contaminant to an ionic liquid sorbent or from the ionic liquid sorbent, the contaminant removal device comprising:
a housing comprising a liquid inlet configured to introduce the ionic liquid sorbent into the housing; and
a packed bed configured to receive the ionic liquid sorbent and rotate about a device axis to centrifugally force the ionic liquid sorbent through the packed bed.

2. The contaminant removal device of claim 1, wherein the housing is a first housing, the device further comprising a second housing within an interior of the first housing,
wherein the second housing is configured to retain the packed bed,
wherein the first housing is configured to maintain a fixed position; and
wherein the second housing is configured to rotate relative to the first housing and about the device axis to cause the packed bed to rotate about the device axis.

3. The contaminant removal device of claim 2, further comprising a motor mechanically coupled to the second housing and configured to rotate the second housing.

4. The contaminant removal device of claim 2, wherein the second housing defines a circumferential bulge radially outward of the packed bed relative to the device axis, wherein the circumferential bulge defines a collection region configured to receive the ionic liquid sorbent centrifugally forced through the packed bed.

5. The contaminant removal device of claim 1, wherein the housing further comprises a liquid outlet, the device further comprising a pitot tube configured to transport centrifugally forced ionic liquid sorbent from the packed bed to the liquid outlet.

6. The contaminant removal device of claim 5, wherein the pitot tube defines a pitot inlet positioned in a collection region radially outward of the packed bed relative to the device axis, and wherein the pitot inlet is configured to introduce at least a portion of the ionic liquid sorbent from the collection region into the pitot tube.

7. The contaminant removal device of claim 6, wherein the pitot inlet comprises an inlet funnel.

8. The contaminant removal device of claim 5, wherein the pitot tube is a first pitot tube, and wherein the contaminant removal device further comprises:
a second pitot tube radially inward relative to the first pitot tube and configured to introduce at least a portion of the ionic liquid sorbent from an overflow region into the second pitot tube; and
a third pitot tube radially between the first pitot tube and the second pitot tube and configured to recirculate the ionic liquid sorbent.

9. The contaminant removal device of claim 1, further comprising a liquid distribution conduit coupled to the liquid inlet and extending along the device axis, wherein the liquid distribution conduit defines a plurality of liquid distribution openings configured to distribute the ionic liquid sorbent received from the liquid inlet to the packed bed.

10. The contaminant removal device of claim 1, wherein the contaminant removal device is a scrubber configured to absorb the contaminant from an air stream into the ionic liquid sorbent, the scrubber further comprising a gas inlet configured to introduce the air stream into the scrubber.

11. The contaminant removal device of claim 10, further comprising a gas outlet configured to discharge a processed air stream from which the contaminant is at least partially scrubbed by the scrubber.

12. The contaminant removal device of claim 1, wherein the contaminant removal device is a stripper configured to desorb the contaminant from the ionic liquid sorbent, the stripper further comprising a liquid outlet configured to discharge a processed liquid stream from which the contaminant is at least partially stripped by the stripper.

13. The contaminant removal device of claim 12, further comprising a gas outlet configured to discharge a gas stream comprising the contaminant at least partially stripped by the stripper.

14. A contaminant removal system, comprising:
a scrubber configured to absorb a contaminant from an air stream into an ionic liquid sorbent, the scrubber comprising:
a scrubber housing;
a gas inlet configured to introduce the air stream into the scrubber housing;
a liquid inlet being configured to introduce the ionic liquid sorbent into the scrubber housing; and
a scrubber packed bed configured to receive the ionic liquid sorbent from the liquid inlet and rotate about a scrubber axis to centrifugally force the ionic liquid sorbent through the scrubber packed bed.

15. The contaminant removal system of claim 14, further comprising:
a stripper configured to desorb the contaminant from the ionic liquid sorbent, the stripper comprising:
a stripper housing comprising a liquid inlet configured to introduce the ionic liquid absorbent received from the liquid outlet of the scrubber into the stripper housing; and
a stripper packed bed configured to receive the ionic liquid absorbent from the liquid inlet and rotate about a stripper axis to centrifugally force the ionic liquid absorbent through the stripper packed bed.

16. The contaminant removal system of claim 14, wherein the contaminant is carbon dioxide.

17. The contaminant removal system of claim 16, further comprising a Sabatier system coupled to the ionic liquid regeneration assembly and configured to generate hydrocarbons using the contaminant.

18. A method for removing a contaminant from an environment, the method comprising:
absorbing, by a scrubber, a contaminant from an air stream into an ionic liquid sorbent centrifugally forced through a rotating scrubber packed bed in the scrubber;
transporting the ionic liquid sorbent comprising the contaminant to a stripper; and
desorbing, by the stripper, the contaminant from the ionic liquid sorbent.

19. The method of claim 18, wherein the desorbing further comprises centrifugally forcing the ionic liquid sorbent through a rotating stripper packed bed in the stripper.

20. The method of claim 18, wherein the contaminant is carbon dioxide, the method further comprising generating, by a Sabatier system, hydrocarbons using the desorbed contaminant.
